(19) United States
(12) Patent Application Publication
(10) Pub. No.: US 2019/0126056 A1
(43) Pub. Date: May 2, 2019

(54) ELECTROMAGNETIC EQUIPMENT FOR INCREASING TISSUE ELASTICITY

(71) Applicant: Constantin VLÅDILA BOGDAN, Bucharest (RO)

(72) Inventor: Constantin VLÅDILA BOGDAN, Bucharest (RO)

(21) Appl. No.: 16/092,777

(22) PCT Filed: Apr. 3, 2017
(86) PCT No.: PCT/RO2017/000005

§ 371 Date: Oct. 11, 2018

(30) Foreign Application Priority Data
Apr. 11, 2016 (RO) A 2016 00258

(51) Int. Cl. A61N 2/02; A61N 2/00

(52) U.S. Cl. CPC A61N 2/02; A61N 2/006

(57) ABSTRACT

The present invention relates to electromagnetic equipment for increasing the tissue elasticity used, particularly in the cosmetic and the medical field. The electromagnetic according of the invention, contains a support (A), a coil-applicator (B) for the application of the electromagnetic field, a filter (C) for filtering the current's harmonics applied to a device (D) generating an electromagnetic field, the ensemble made up of the device (D), the filter (C) and the coil-applicator (B), being constructed to generate inside the coil-applicator (B) in an X area, an uniform electromagnetic field, with the induction between 0.7-0.8 mT, with a variation between 5-10% in the area subjected by the electromagnetic field, on a distance of 1 cm around the same point, on a larger volume in the treated areas, produced by a current of 400 mA, of a frequency between 7 and 8 Hz, preferably 7.69 Hz, with field lines perpendicular on the surface of the skin, and wherein the coil-applicator (B) in which it applies an uniform electromagnetic field at right angles to the localized area next to their cell tissue, it is composed of two magnetic cores, one inner (5) and one outer (6) in the form of a circle and a housing (7) around each core (5, 6) being wired a small coil (9) and, respectively, a large coil (10), coupled in series with one another, so it can allow an induction of 0.6-0.8 mT in the treatment area, and a uniform magnetic field perpendicular to the treated surface, for example, the skin, the output of the large coil (10) being connected to the input of the small coil (9) and the input of the large coil (10) and the output of the small coil (9) being electrically connected to the small unit (D) electronically generating the electromagnetic field through the filter (C) so it can be filtered the harmonics current flowing in the device (D).

The present invention relates to an electromagnetic equipment for increasing the tissue elasticity used, particularly in the cosmetic field, namely, to reduce wrinkles, to regulate secretion of the sebaceous glands, to prolong the time in which it has to be re-injected hyaluronic acid in patients with wrinkles but also to combat baldness and hair loss, causing the appearing of myofibroblasts. In addition, due to the fact that an increase and regulation of microcirculation and tissues are also produced, as well as a reinforcement of cell, the present invention can be used in the medical field, with applications in:
Nerve regeneration, for example Parkinson, dementia, depression, Alzheimer, nerve regeneration in ophthalmology;
dry eye syndrome, dry mouth, nasal secretion regulation;
treatment of burns and wounds;
treatment of hemorrhoids and varicose veins;
treating genital diseases of the cervix, ovary and testicles;
reinforcing tissue of ligaments represented by articular disks, but also in other applications involving the improvement of elasticity and, also, growth, regulation of the tissues microcirculation, for example, Otorhinolaryngology (ORL)—rhinitis, sinusitis, loss of hearing, smell, taste, seeing (hyperopia, myopia, presbyopia);
reducing myopia;
slows the advancement of cancer by strengthening the healthy tissue around the cancer. So the advancement is slower and it does not become malignant;

It has been shown that each normal or pathological event which has occurred in any organism, cause changes in the electromagnetic field generated by that organism. Based on these principles they were built devices that monitor heart and brain activity, or by which one can determine with precision ovulation. James Oschman was the one who proved that muscle activity generates electromagnetic pulses that stimulate cellular regeneration, starting from the attraction of mesenchyme undifferentiated cells and that any condition such as an injury caused by surgery determinates a modification of the magnetic field in the tissue trauma region. In other words, changing the number of capillaries is accompanied by an increase of the magnetic “resistance” of adjacent tissues. This increase is also determined by any metallic local implant that disrupts or prevents the normal magnetic field from regeneration which results from Schumann magnetic frequencies and biological cellular oscillations. From the aspects described above, it can be concluded that the biological integration, prevented by increasing the magnetic resistance in that portion due to the tissue injury, the tartar, the microbes presented in that area and also, due to the insertion of any metallic implant can be, however, accelerated by bringing the regenerated magnetic field to normal values.

Sicken and Walker have demonstrated that the frequencies of 2 Hz, 25 Hz and 50 Hz stimulate nervous regeneration, which is useful in implantology if hypoesthesia occurs following a surgical procedure. Sisken and Walker have also demonstrated that a 7 Hz frequency stimulates osseous regeneration and that a 10 Hz frequency stimulates ligament regeneration, which is for instance useful in par odontology for lowering teeth mobility. Sisken and Walker have also demonstrated that the 15 Hz, 20 Hz and 72 Hz frequencies stimulate the reformation of capillaries, this action being necessary after any surgical intervention involving osseous or soft tissue graft. Herbert Frachlich demonstrated that an assembly of cells forming a tissue or organ has a specific frequency that regulates the physiology of said organ and if a big number of cells are affected, then the frequency cannot be emitted any longer and the disease or dysfunction occurs.

Those described above show that a healthy organ contributes to maintaining the health in its neighboring region, and a diseased organ cannot do this any longer and suitable steps should be taken to remedy the situation. When these measures must be applied in oral cavity in particular, attention must be in that the natural regenerating field is reduced, since the microbes and minerals extant in the tophus composition “steal” from the regenerating field intensity in order to mineralize themselves. It is known that the phenomena of healing by magnetic oscillations occur at low amplitudes of the field, with a magnetic induction of about 10⁻⁹ to 10⁻¹⁰ Tesla.

There are known cosmetic devices comprising a device to generate an electromagnetic field to speed up the recovery of the skin tissues comprising devices for generating an electromagnetic field with a frequency, preferably of 7, 692 Hz and electromagnetic radiation of 0.75 mT in the oral cavity, used in cosmetic treatments and hair overgrowth. The device consists of a circuit of electromagnetic field generation of extremely low frequency, and is connected with a local application device for producing of an electromagnetic field of extremely low frequency in the oral cavity, and for a constant sinusoidal electric current of extremely low frequency, the circuit of electromagnetic field generation is composed of a quartz oscillator that generates a rectangular signal of initial frequency at high precision, which is successively divided through an integrated circuit whose output is obtained at a reduced frequency, from an integrated filter Butterworth typed circuit, with which the rectangular signal is converted into sinusoidal signal, from a signal attenuator in eight steps, in order to provide a current in the area of 0.25 mT-2 mT, every step of it resulting in an increase of 0.25 mT of the induction magnetic field obtained between some polar parts of the device for the local application of the electromagnetic field, and from a constant current source; the device for the application of electromagnetic field in the oral cavity is a tweezers, made of permalloy with adjustable opening between its ends, ends which have, in their final part, some polar parts which apply to the area of interest, being placed in the middle of the tweezer a coil that generates magnetic field of magnitude and shape imposed by the electromagnetic field generation of extremely low frequency. (Patent No. RO 128805 B1 from 30.03.2014)

The disadvantages of these devices consist in that the device for local application is relatively difficult supported by the patients, particularly by those who have a high sensitivity.

Also, the devices known in the field generate electromagnetic pulses of extremely low frequencies with intensities and, sometimes, amplitudes, significantly lower than those attributed to the terrestrial magnetism. However, such electromagnetic fields include a current component and display oscillations for the same reason, that's why effects at a cellular level of these devices are not expected and are also supported relatively difficult by patients, particularly by those who have a high sensitivity and don't allow an application rather than on small areas, which increases the duration of treatment. For example, the international patent application WO 2009/04215 A1 discloses an electronic device designed to accelerate the healing tissue in the knee, through the electromagnetic field with low frequency. The device is part of a system designed to heal tissues and bones with different conditions, and is provided with an electric circuit that controls the pulse of the magnetic field.

The technical problem that the cosmetic device solves, according to the present invention, consists in reassuring that it is generated an uniform electromagnetic field perpendicular on the treatment area of the skin which can lead to the regain of the tissue elasticity, the increase and regulation in their microcirculation and also tissue at the ligaments level.

An electromagnetic equipment for increasing tissue elasticity used in the cosmetic field, according to the invention, contains a support, a coil-applicator for the application of the electromagnetic field, a filter for filtering the current's harmonics applied to a device generating an electromagnetic field, the ensemble made up of the device, the filter and the coil-applicator being constructed to generate inside the coil-applicator in an X area, an uniform electromagnetic field, with the induction between 0.7-0.8 mT, with a variation between 5-10% in the area subjected by the electromagnetic field, on a distance of 1 cm around the same point, on a larger volume in the treated areas, produced by a current of 400 mA, of a frequency between 7 and 8 Hz, preferably 7.69 Hz, with field lines perpendicular on the surface of the skin, and wherein

According to another aspect of the invention, the coil-applicator in which it applies an uniform electromagnetic field at right angles to the localized area next to their cell tissue, it is composed of two magnetic cores, one inner and one outer in the form of a circle and a housing around each core being wired a small coil and, respectively, a large coil, coupled in series with one another, so it can allow an induction of 0.6-0.8 mT in the treatment area, and a uniform magnetic field perpendicular to the treated surface, for example, the skin, the output of the large coil being connected to the input of the small coil and the input of the large coil and the output of the small coil being electrically connected to the small unit electronically generating the electromagnetic field through the filter so it can be filtered the harmonics current flowing in the device.

According to another aspect of the invention, core on which is wired the small magnetic coil, has a diameter of 15-33 cm, preferably 30 cm, and a thickness of 0, 3-0.43 cm, preferably 0.4 cm, and the magnetic core on which is wired the large magnetic coil, has a diameter of 16-34 cm, preferably 31.5 cm and a thickness of 0.3 0.43 cm, preferably 0.4 cm.

According to another aspect of the invention, the small coil is made of small enameled copper wire with a diameter of 1-1.5 mm, preferably 1.2 mm, with a number of turns between 45-50, preferably 48 turns, with a wired width of 6 cm and the large coil is made of large enameled copper wire with a diameter of 1-1.5 mm, preferably 1.2 mm, the number of turns between 650-700, preferably 680 turns, with the wired width of 6 cm.

According to another aspect of the invention, the assembly of the magnetic cores, the coils and the filter are fixed in a housing made of two half-housings which consists of stidotextolit material, which may or may not be covered by textile material for the protection of the skin.

According to another aspect of the invention, the assembly of the magnetic cores coils, the filter and the device is embedded in the housing.

According to another aspect of the invention, the device is a generator for sinusoidal current of low frequency in the range of 2 Hz-25 Hz, with the level of the output current of 400 mA, the maximum output voltage of 24V, and it is built around a microprocessor which generates current signals of low frequency, which generates an electromagnetic field of intensity and uniformed induction over a distance of 1 cm around the same point, on a volume larger on areas in which would be positioned the skin, of 0.75 mT-0.8 mT, and a direct digital synthesizer adapted to directly generate accurate sinusoidal signal with a range of 2-50 Hz, preferably 7.76 Hz, the high accuracy and stability led by the processor, the device comprising a unit of processing, synthesizer (DDS), a block digital amplifier by reaction of current, an audio block amplifier, a clock block for real-time, a memory block, an on/off unit and a supply unit.

According to another aspect of the invention, the microprocessor is implemented as dedicated software that has as functions:
the limitation of the usage period of the device according to the invention at the period prescribed by the medical specialist, but no more than two hours daily for a maximum of 100 hours for cosmetics to 200 hours maximum for medical applications, so it is ordered;
interruption of supply or signal generation for 12-24 hours after using for an hour or two hours daily the equipment according to the invention, depending on the desired application: cosmetic or medical;
the total interruption of the supply or signal generation after using for a maximum of 100 hours for cosmetics or up to 200 hours for medical applications
refueling the equipment or restarting the signal generation according to the invention, by a user code supplied by the operator through a software implemented on the microprocessor.

According to another aspect of the invention, the equipment from the claims 1-7 is used for the growth and regulation of microcirculation tissues in:
nerve regeneration, for example Parkinson, dementia, depression, Alzheimer, nerve regeneration in ophthalmology
dry eye and dry mouth syndromes, adjustment of the nasal secretion
treatment for burns and wounds
treatment for hemorrhoids and varicose veins
treating genital diseases of the cervix, ovary and testicular, tissue reinforcement at the ligaments level represented by the articular disks.
regulating the tissues microcirculation used in rhinitis, sinusitis, loss of hearing, smell, taste, sight.

The equipment, according to the present invention has the following advantages:
accelerate the repairmen of tissues;
The person's device is well supported during the treatment of patients without any effort from their part;
relatively simple architecture;
operational safety.

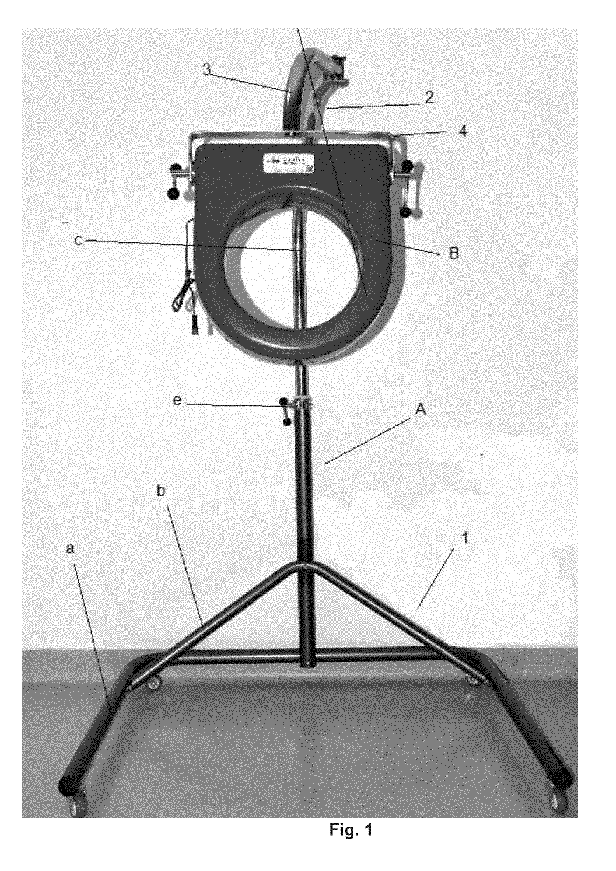

There are described next three embodiments of the electromagnetic equipment for cosmetics and medicine according to the invention, in connection to FIG. 1 . . . 16, which represent:

FIG. 1—general view of the equipment according to the invention;

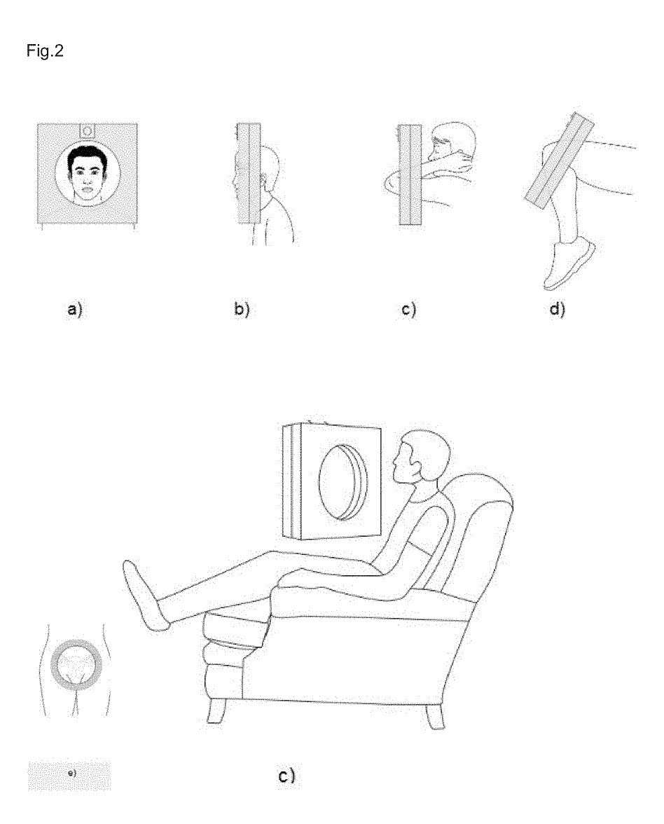

FIG. 2, a), . . . , h)—usage of the equipment according to the invention;

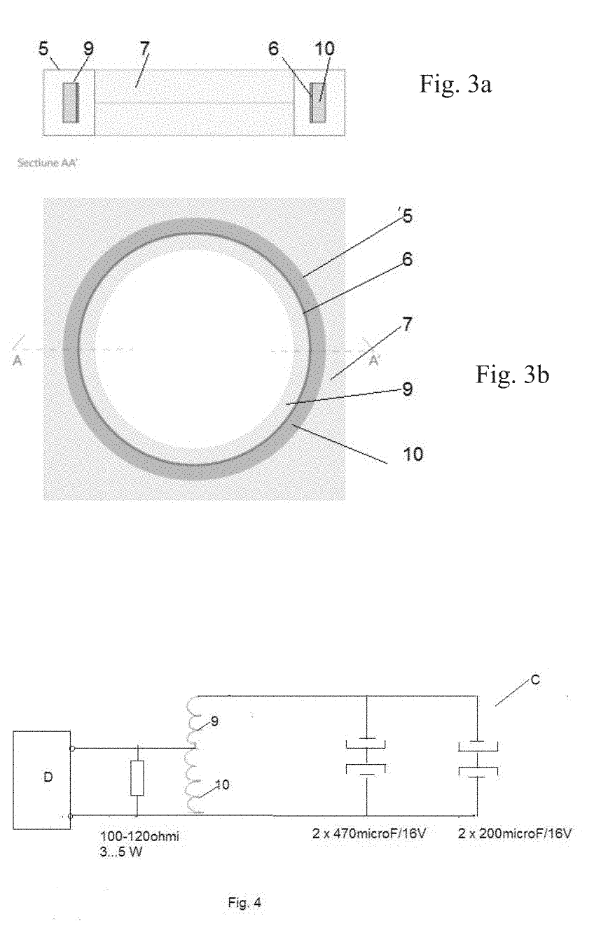

FIG. 3—a) transversal section view through the coil applicator;
b) a longitudinal sectional view through the coil applicator;

FIG. 4 is a schematic circuit diagram of the assembly of the coil applicator, the filter and the electromagnetic field generation device according to an embodiment of the electromagnetic equipment;

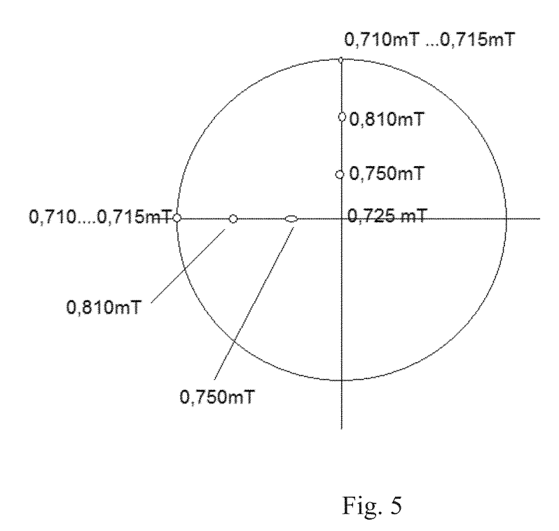

FIG. 5 is the distribution of the electromagnetic field, from the inside part of the coil according to an embodiment of the electromagnetic equipment;

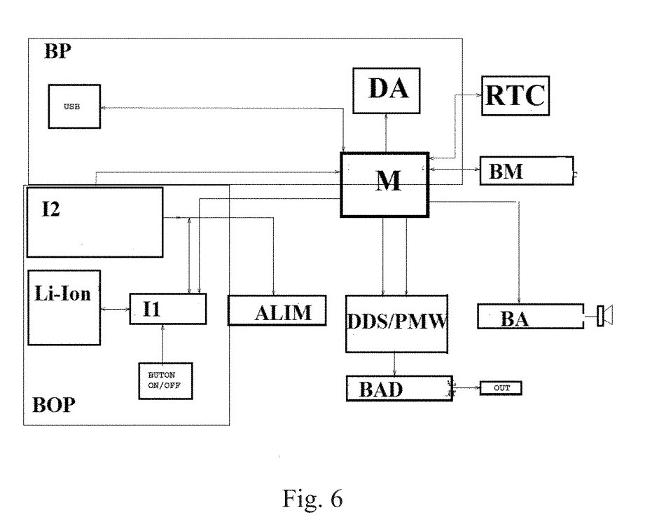

FIG. 6 is a block diagram of the device for generating the electromagnetic field, according to an embodiment of the electromagnetic equipment;

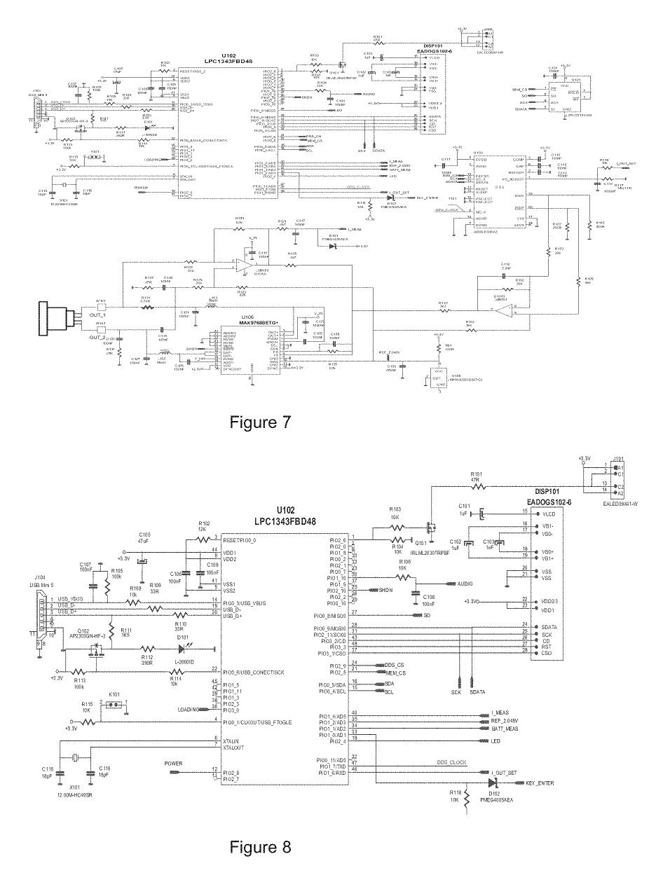

FIG. 7, the detailed electronic scheme of the unit for generating the electromagnetic field, according to an embodiment of the electromagnetic equipment;

FIG. 8 is an electronic scheme of the electromagnetic field generation according to an embodiment of the electromagnetic equipment;

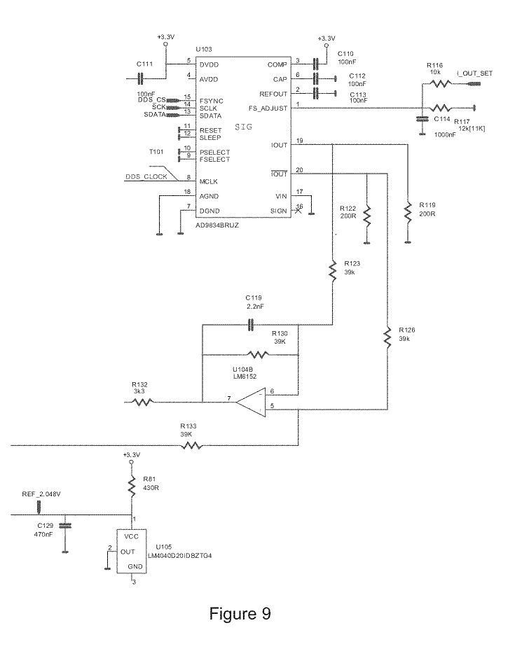

FIG. 9 is an electronic scheme of sinusoidal generator device for generating electromagnetic field according to an embodiment of the electromagnetic equipment;

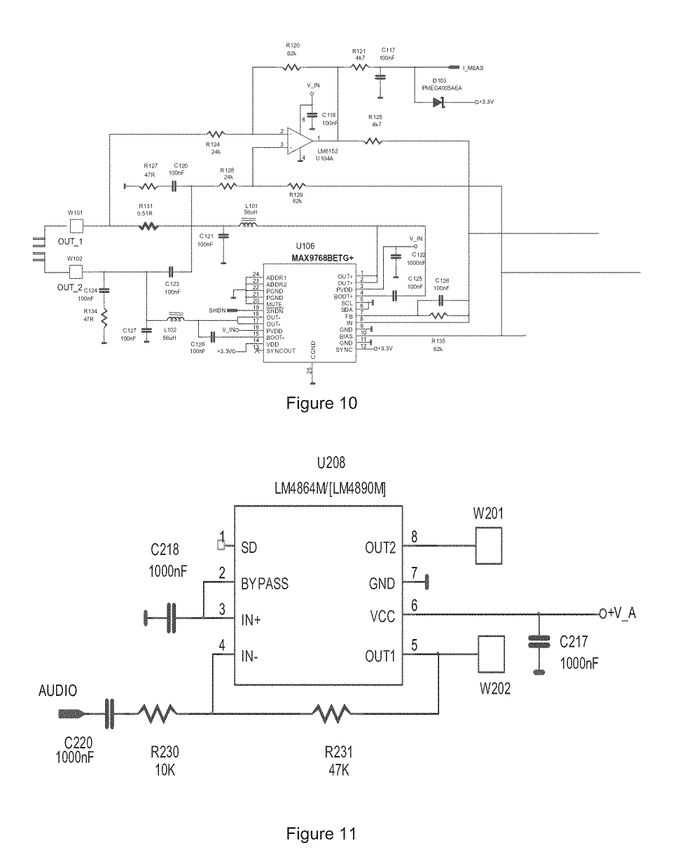

FIG. 10 is an electronic scheme of amplifier digital electromagnetic field generation device, according to an embodiment of the electromagnetic equipment;

FIG. 11 is an electronic scheme of audio amplification device for generating electromagnetic field, according to an embodiment of the cosmetic device;

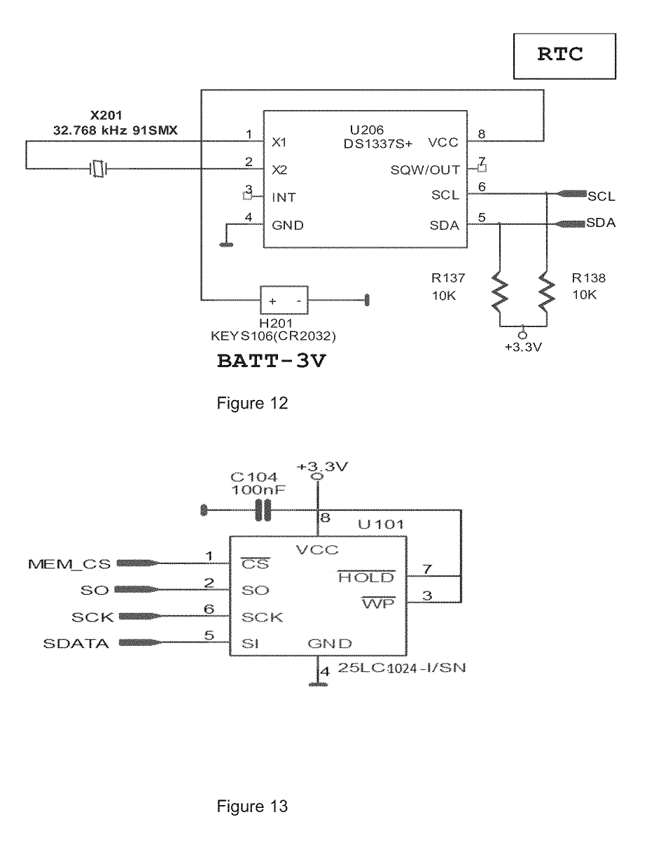

FIG. 12 is an electronic scheme of real-time clock block of the device for generating electromagnetic field, according to an embodiment of the electromagnetic equipment;

FIG. 13 is an electronic scheme of block diagram memory generating electromagnetic field, according to an embodiment of the electromagnetic equipment;

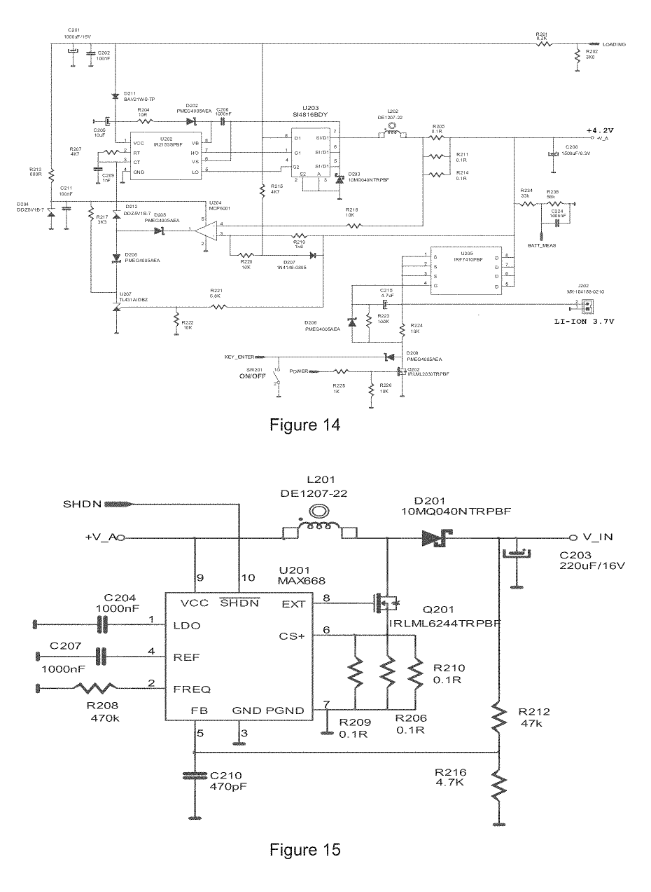

FIG. 14 is an electronic scheme of diagram start/stop block device for generating electromagnetic field, according to an embodiment of the electromagnetic equipment;

FIG. 15 is an electronic scheme of power device for generating electromagnetic field, according to an embodiment of the electromagnetic equipment;

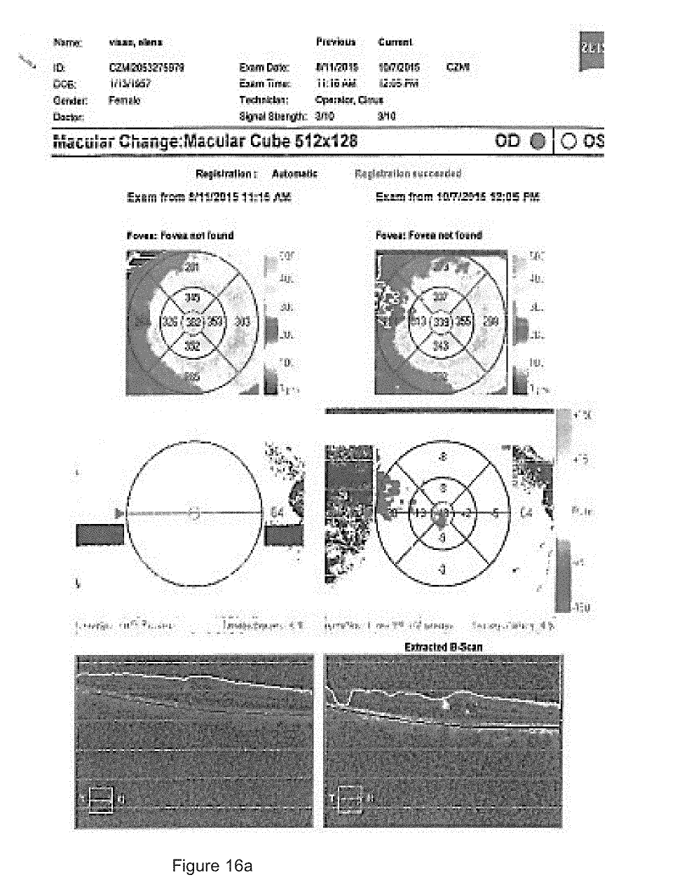

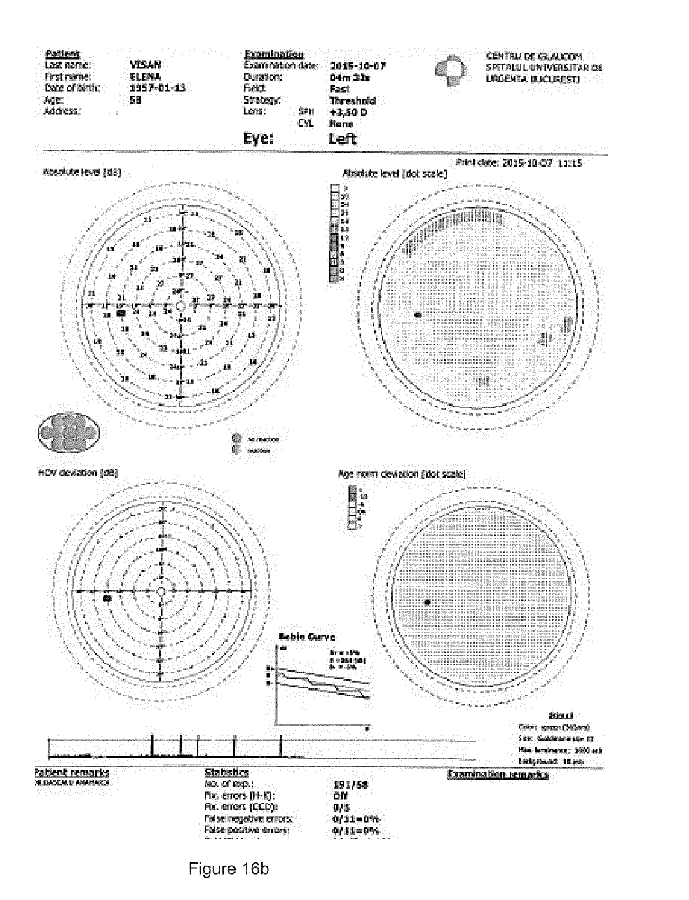

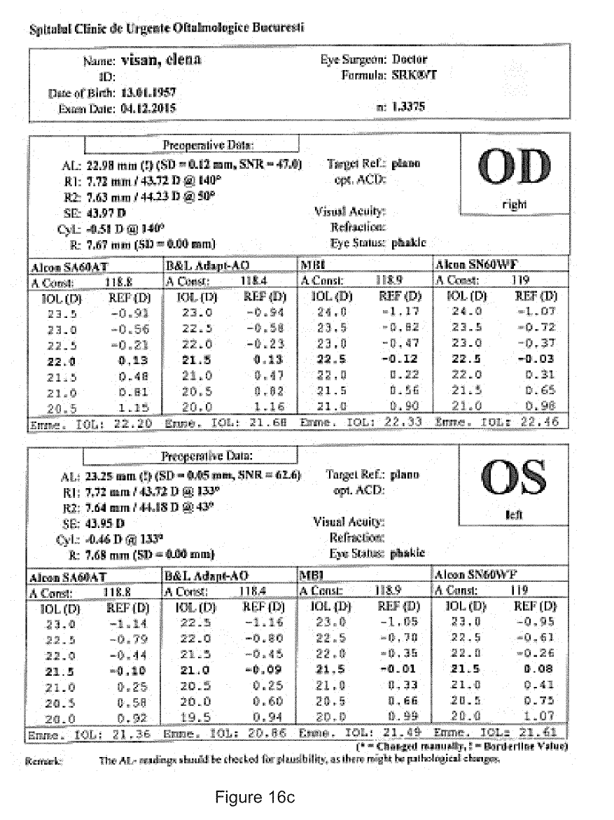

FIG. 16 a) . . . c) presents images made on patients before and after treatment using the equipment, according to the invention, in ophthalmology.

Electromagnetic equipment to increase tissue elasticity for applications in cosmetics and medicine, according to a first embodiment of the invention comprises a support A, a coil applicator B for applications in the electromagnetic field in the desired area, a filter C which filters the sinusoidal current applied of the harmonics and a generating device D for generating an electromagnetic field which is located in a box not represented in the figures. The ensemble composed of the device D, the filter C and the applicator B are designed as to generate inside the coil applicator B an uniform electromagnetic field, with the induction between 0.7-0.8 mT, with a variation of 5-10% in the subjected area of the electromagnetic field, on a distance of 1 cm around the same point, on a larger volume in the areas of application, produced by a current of 400 mA, with the frequency between 7 and 8 Hz, preferably 7.69 Hz, field lines being perpendicular to the surface of the skin.

The A support is used to secure the coil applicator B, so that the user can sit in a comfortable position during the cosmetic and/or medical treatment. Also the A support allows the usage of the coil applicator B in an overhead position, which facilitates the usage of the equipment according to the invention to treat baldness and hair loss, but also the head treatment: nerve diseases, ophthalmology, problems with smell, etc.

The A support is made of a part 1 of maintaining stability and a curved part 2 that sustains an adjustable part 3 where the coil applicator B is caught. The coil applicator B binds to the A support with a U-shaped bar 4 which is provided with the fastening screws. These screws allow the coil applicator B to move into the desired position, for example at the head level, face, knee, etc.

Part I of maintaining the stability contains a U-shaped bar a and a V-shaped bar b. The U-shaped bar a and the V-shaped bar b are attached to the curved part 2 through the screws and nuts or any other ways equivalent to these.

The curved part 2 contains a fixed c bar naked inside to which it is attached the adjustable part 3. The c bar and the adjustable part 3 are held together by an adjustable e screw which is adjustable in height for the curved part 2. This allows the coil applicator B to positioned at any height, the height desired by the patient. The adjustable bar 3 is made up of three sections d1, d2 and d3. The segment d3 is the part that is held on with the adjustable screw e by the part 1 of maintaining stability and it is an adjustable extension of this. The segment d2 has an oblique shape and is continued with the vertical segment d3 to which is attached the U-shaped adjustable support bar B.

The bars a, b, c, d of the fixed support A are metallic, preferably aluminum, so they can be transported easily.

The adjustable part 3, as stated above, it consists of a U-shaped bar 4 that is provided in the sides with some clamping screws. These screws allow the coil applicator B to be fixed in the desired position, for example on the head level, face, knee, etc.

According to another embodiment, not shown in the drawings, the coil applicator B may be attached directly to the ceiling with the help of a strip of cloth fabric fastener provided with the coil applicator B and a system for adjusting the position of the coil applicator. The system can be, but not limited to, a clamp known per se. The position adjustment system may be, but not limited to, a clasp type device known per se.

Furthermore, according to another embodiment, not represented in the drawings, inside the support a there may be one or two linear electric motors, in small dimensions, which can be ordered by radio —remote controlled—in order to adjust the height and position of the coil applicator B. One of the engines is fixed at the adjustable part 3 to adjust the height used by the coil applicator B. The second motor is fixed on the attached bar 4 to adjust the position of the coil applicator B. Power engines is achieved by batteries.

The coil applicator B generates a uniform electromagnetic field perpendicular on the located area next to their cell tissue.

The coil applicator B includes two magnetic cores, an inner one 5 and an outer one 6, which are mounted within a housing 7 made up of two semi-housing, respectively lower and upper.

In around each core 5 and, respectively, 6 are wired a small coil 9 and, respectively, a large coil 10. According to a concrete embodiment, the core magnetic 5 on which is wired the small coil 8, is made from galvanized steel of 4 mm with a length of 90-95 cm, preferably 94.5 cm, a width of 6 cm and a diameter of 29-33 cm, preferably 30 cm.

The magnetic core 6 on which is wired the large coil 10 has a diameter of 30-34 cm, preferably 31.5 cm, a thickness of 0.4 cm and a length of 95-100 cm, preferably 97.5 cm.

The magnetic cores 5 and 6 are made of galvanized steel sheets.

The small coil 9 is made of enameled copper wire with diameter of 1-1.5 mm, preferably 1.2 mm, number of turns between 45-50, preferably 48 turns, wire width of 6 cm. Turns of the small coil 9 are wired to the whole width of the sheet over the magnetic core 6.

The large coil 10 is made of enamel copper of 1-1.5 mm diameter, preferably 1.2 mm; the number of turns is 650-700, preferably 680 turns, with width of 6 cm.

After the 680 turns of the large coil 10, the core magnetic 5 with the small coil 9 is inserted into the core 6 with the large coil 10. This makes electrical connections as follows: the end of the large coil 10 sums up with the beginning of the small coil 9, the beginning of the large coil 10 and the end of the small coil 9 connects to the attenuation filter C of the current harmonics that connect through electrical connectors to the device D for generating the electromagnetic field.

The housing 7 is made, preferably, of sticlotextolit material, which can or cannot be covered by textile material, for skin protection, for example.

The current harmonics filtration filter C flowing to the device D, has a construction known per se and does not require a detailed description in this presentation.

According to another embodiment, the ensemble formed from the magnetic cores 5, 6, the coils 9, 10, the filter C, and the device D is fixed 10 to the housing 10. This construction allows easier work with the equipment, according to the invention.

The bioactive treatment with electromagnetic field of extremely low frequency emitted by the device D is made by the coil applicator B.

The device D for the application of the electromagnetic field is the generation of electromagnetic field of a very low frequency, and is thus built as to generate inside of the mouth guards A or B or F a uniform field of induction comprised in the range between 0.7-0.9 mT, with a variation between 5-10% in the area subjected to the electromagnetic field, a distance of 1 cm around the same point on a larger volume in areas that would be positioned dental root or implant, produced by a current of 200 mA, ranging between 7 and 8 Hz, preferably 7.69 Hz

In order to obtain the above parameters, according to an embodiment, the device D is a generator of sinusoidal low frequency current in 2 Hz-25 Hz range, output level of 200 mA, maximum output voltage of 24 V RMS. Power is achieved through a battery of Li-ion accumulators.

According to an embodiment, the device D is built around a microprocessor M that generates current signals of low frequency.

With the device D, the field frequency ELF is only one and is already fixed, and its uniform induction in the area/target areas is substantially 0.75 mT . . . 0.9 mT, so, as you may be somewhat greater at the mouth guards level, will be potentially up to 3 mT, if you use the current of 400 mA for the depth of the bone.

Normally, for generating sinusoidal signal would have several possibilities:

a. Using a high-frequency generator and programmable frequency dividers for frequency in the form of rectangular signal and then filtered to get sinusoidal shape. This generates some signals with amplitude depends on the tolerance of the components in filter and discrete resolution due to the divider. This variant is not satisfactory.
b. Building a sinusoidal oscillator directly at work frequency but it wouldn't have the precision and stability.
c. Using direct digital synthesis variant, variant that is preferred and will be detailed below.

Thus, in order to obtain a continuous signal constantly and sinusoidal of extremely low frequency, according to one embodiment of circuits of the device D, it contains a Digital Synthesizer of constant current DDS adapted to generate directly sinusoidal signal precisely with a variation from 2 to 50 Hz, which is set at a frequency of between 7 and 8 Hz preferably 7.69 Hz Signal generated by DDS has a high accuracy and stability led by processor M.

According to an embodiment, D contains a processing block BP, the synthesizer DDS containing a block generator sinus PMW, a digital amplifier block BAD with current reaction, an audio block amplifier BA, a clock block RTC for real time, a block of memory BM, an on/off switch block BOP and a power unit ALIM.

The processing block BP contains the microprocessor M, a display device DA and some connection jacks USB. The microprocessor M generates the following signals:
clock signal SCK for serial communication;
data signal SDATA for serial communication;
signal CSD to pin nr 37, selecting the display device DA during communication;
signal MEM CIP SELECT selecting memory during communication with it;
signal SO selecting read-write memory;
signal DDS_CS selecting the write signal DDS;
database signal SDA for communication I2C at RTC;
clock signal SCL for I2C communication at RTC;
signal Power switch of auto maintenance switch on (active on 1);
signal I_out_set rectangle with variable fill factor for the generator PWM for command output current level;
clock signal DDS_CLOCK for synthesizer (DDS);
signal LED from flagging operation through the intermittent lighting up of the led;
read signal Audio from memory and filtered from the generator PWM out of pin 30 of the microprocessor M;
signal Backlight coming out of the pin 1 of the microprocessor M and to light backlight display device DA

input signals in the microprocessor M:
signal (I_meas) measurement of output current that comes in the form of alternative and is numeric filtered in the microprocessor (M) and measured;
signal (Bat_meas) of constant voltage divided from the supply voltage for battery monitoring;
signal (Key_enter) given by the on/off switch to turn off the device
signal (Ref_2048) of the reference voltage for measurement current

Having in view of requirements of communication with a computer, it can be used, for example, an ARM Cortex-M3 microprocessor that does not require programming, BOOT LOADER for loading making it via USB like loading a memory stick directly under Windows. This microprocessor must have communication systems such as I2C and SPI and miniature capsule (LQFP48).

Sinusoidal signal generated by DDS enters the digital amplifier blocks BAD and audio BA with an adjustable constant current which can vary from 1 to 400 mA but output will have only one preset value. Both the frequency and current are continuously controlled by M. The outputs of the amplification filtered blocks are applied to the relevant terminals of the device D that is connected functionally to coils 8, 9 and respectively, 19 based on what mouth guards A, C, and/or F are used at the time.

Integrated circuit of the synthesizer DDS, shown in FIG. 24, has two registry of frequency Rfreq and two registry of phase Rphase where you can write two frequencies and two phases in a system of interface SPI. In this case, set only the frequency register Rfreq. Sinus table is in SIN ROM and his running will be done at the written frequency of the dock generator RTC gave by the microprocessor M. For example, using a dock generator integrated RTC 7 W-25,000 MBAT of 25 MHz or dock signal is obtained with processor M by splitting the quartz crystal of 12 MHz through 210 and get 11718.75 Hz which is the reference dock signal of DDS synthesizer.

Frequency resolution of the output current is the ratio between clock frequency and the frequency register 228

Rez=11718.75/228=0.000043655 Hz.

The sinus block generator PMW consists of some modulators involving current reaction and limitation of PWM-signal value. It uses the system to compare a triangular high-frequency signal (120 Khz) with sinusoidal signal generated by DDS, such that the input signal is converted to a rectangular symmetrically signal. This symmetrical signal is amplified and added with the current reaction signal and applied to block digital amplifier BDA. Thus it ensures the voltage reference for the attachment of reference sinusoidal signal in digital amplifier blocks BAD and respectively audio BA and in microprocessor M in the measurement.

In the block digital amplifier BAD with current reaction, the input signal from the sinus block generator PMW is applied to pin 8 (IN) reference signal pin 10 (BIAS). Output current passes through a resistance reaction and measure R131. The voltage produced is amplified of block BA and represents the current value of output for measurement (I_MEAS) and in the meantime added through R125 with input signal, creating the current reaction. The current output is obtained from out1 and out 2 by filtering passes down with filters and 1101, c102, 1121 and c127.

BAD block can contain a linear amplifier, but its efficiency is 56% maximum when the output voltage has excursion equal to the supply voltage, but in this case, where the resistance of the mouth guards has various values, it follows that the efficiency is between 0% and 56%, which is not allowed because it cannot ensure the autonomy of operation and evacuation.

Such it uses linear amplifier version in class D (switch), for example the MAX9768. The power requirement is 3.6 W RMS in output.

In the audio amplifier block BA audio signal resulting from the microprocessor M is applied at the input block amplifier BA, where is boosted 4.7 times and plugged into the speaker through the terminals w201 and w202.

Having the low supply voltage (3.7V), it chooses for block BA a small voltage in topology BTL low power. You can choose for example integrated circuit LM4864.

The real time clock RTC is made with an usual integrated circuit work, e.g. DS 1337 and who communicate with the microprocessor M and is powered permanently with 3 volt auxiliary battery which ensures the independence of at least two years.

Setting the date and time and reading is done on the microprocessor M.

Memory block WB has in composing an integrated circuit, i.e. 24 LC1024 where you can save 1024 Kbyt. The audio file is saved from the microprocessor M and converted into PWM signal that by filtering represents memorized audio signal. Choose a memory of 1024 Kbps because you need to load the audio file in it that has minimum 200 Kbytes and at the same time it must be memorized treatment history and any information. For example, you can use memory 25LC1024.

The on/off switch block BOP contains a floor switch I1 and a floor loading I2. The electronic floor switch I1 is made with mosfet type U205, transistor of auto maintenance q202 and adjacent resistors. The pressing starting stop by D209, a resistance R223 that polarize their circuit grid U205 opens coupling the battery to device D.

After resetting the first thing microprocessor does is signal generation power that opens transistor q202 and auto maintenance open the circuit U205.

To stop, by pressing switched off the voltage from pin 33 of microprocessor M is cancelled by a diode D102, which means off to signal processor M which makes life-saving functions of the parameters and then cancel the signal Power and the device stops.

Floor loading 12 is achieved by clock generator PWM U202, semi bridge of mosfet U203, voltage regulator U207 and current limiting circuit U204.

The introduction of charger is supplied clock generator PWM U202 and creates signal LOADING. At exit filter L202 4.2 V voltage occurs which is controlled via the generator PMW U207 modulating created signal PWM. Microprocessor M being supplied with this voltage starts and detects the signal LOADING.

Then open the electronic switch CE and is coupled the accumulator to charging.

Load current is measured on the resistant group R205, R211 and R214 and the generator PMW U204 and adjusted by PWM modulation at 2A value by reducing the voltage of 4.2 V.

Under the current of 2 A battery voltage grows and at achieving the value of 4.2 Volt, voltage regulator takes signal modulation PWM to maintain voltage 4.2 V, which is reflected in the reduction of load current up to 0.

The power supply ALIM incorporates a source of 14 volts which is a Boost Converter type and is made with an integrated circuit U201 and transistor Q201. The output power is limited to 5 W by choosing the value of sense resistances, R206, R209 and R210. ALIM block also contains a 3.3 volt source performed with an integrated circuit, for example MCP1804 which has the minimum input voltage 3.4 volts.

Basically, it uses the supply voltage of a Lithium-Ion cells to ease loading system in case you place several batteries in parallel and not in series since then should be balancing circuit. ALIM block must be a source of constant current limiting when the voltage reaches 4.2V. Battery capacity is determined according to the maximum output power, efficiency and autonomy of operation. For global efficiency of 80% and a minimum voltage of the power supply 3.5 V from calculations resulting an energy of 5 Wh.

Must be ensured a stream of 1.42 Ah. For a greater autonomy of two hours you use two batteries connected in parallel with each capacity 2 Ah.
a. for 3.3 V supply is choose a linear source LD (with little voltage drop at saturation), because the input voltage is between 3.5 and 4.2 V (do not justify source commutation).
b. to supply power floor it takes 14 V and choose Variant Boost Converter, for example MAX668.

The microprocessor M has implemented dedicated software which has the function of limiting the period of the device according to the invention to period prescribed by the medical specialist, but no more than two hours a day, up to 100 hours for cosmetic applications and up to 200 hours maximum for medical applications. Also through the software implemented on the microprocessor M, it orders the closure of supply after using for one hour or two hours of the equipment according to the invention, depending on the desired application: cosmetic or medical.

The implemented software on the microprocessor M orders:
limiting the period of the device, according to the invention, to the period prescribed by the medical specialist, but no more than two hours a day for maximum 100 hours for cosmetics or for maximum 200 hours for medical applications, which means that it is ordered;
The interruption of 12-24 hours of power supply or signal generation after using for one hour or two hours daily the equipment according to the invention, depending on the desired application: cosmetic or medical;
the total interruption of power supply or a signal generation after using for a maximum of 100 hours for cosmetics or up to 200 hours for medical application;
refueling the equipment or restart the signal generation, according to the invention for a new package of 100 hours after introducing a user code supplied by the manufacturer via a client software application related the generator's microprocessor and implemented in it.

The method of the tissue regenerator used to improve the elasticity and to improve the increasing and regulation of the tissues microcirculation, for example to reduce wrinkles and for other cosmetic treatments as well as for the treatment and prevention of diseases, is achieved by exposing the electromagnetic field obtained with the device, according to the invention, and consists in the exposure to the electromagnetic field parameters obtained by them daily, for one hour each for cosmetic applications, respectively, for two hours each, for medical applications, for 50-100 hours for cosmetic applications, and for 200-300 hours for medical applications.

The results of the applications testing on patients

A. In the Cosmetic Field

The growth treatment in tissue elasticity through the equipment, according to the invention, has been established for the purpose of this study, to a number of exposures of minimum 50 meetings per one hour each.

Due to the small size of the equipment and to the anatomical adaptability and the height and position adjustment of the coil applicator B, patients were able to stay relaxed, engaged in a mild and pleasant activity (reading a book, watching a TV program). It is recommended a daily period to the exposure of electromagnetic field with a condition that in between two consecutive meetings it cannot be a distance greater than 3 days.

The exposure to the electromagnetic fields acts through a mechanism of stimulation and adjustment through of the appearance of the myfibroblast at a tissue level and for this reason it has been recommended that the treatment with the device, according to the invention, both as an element for prevention and as a mechanism for proper disposal of wrinkles unsightly

From the point of view of prevention in order to preserve the results of the initial cell regeneration, it has been recommended to repeat the average number of sessions annually. To the half numbers of meetings, the assessment protocol repeats itself through objective methods (Dermascan analysis), on the treatment areas.

A third set of measurements was taken at the end of the number of meetings allocated to treatment. Also, at the end of the treatment, the patients repeated the series of photographs for the subjective identification of changes in appearance.

It has been registered the measurements which have been saved for a subsequent comparative analysis (first-intermediate-final).

Clinical case 1. Patient A. P., in the age of 40, male, was submitted on Feb. 19, 2015 at a consult and an ultrasound measurement using the Dermascan. It has been registered the following values in areas of interest. We chose three areas of verification according to the grid areas of interest, according to Table 1 and 2.

After a total of 40 meetings, the dermatological measurements were remade on the areas of interest and were the following changes at the derma level:

After 40 sessions of the equipment according to the invention, placed in an interval of 3 months, with a repetition of sessions of 2-3 days, we see an increase in cell mass on 2 of the 3 areas measured and an increase in the cellular mass density from 6.15 to 8.28, Table 3-Table 6.

TABLE 5

| T0 | T1(40) | T total(After-Baseline) |
|---|---|---|
| 6.15 | 8.28 | 35% |

Clinical case 2. Patient A.Z., at the age of 37, female, was submitted on Jan. 29, 2015 to a consult and a ultra sonographer measurement using Dermascan. It has the following values in areas of interest. We chose three areas of verification according to the grid areas of interest, Table 7 and Table 8.

TABLE 11

| T0 | T1(40) | T total(After-Baseline) |
|---|---|---|
| 6.15 | 8.28 | 32% |

In conclusion, the equipment according to the invention, acts simultaneously on all tissues from the facial area resulting in increased elasticity by reinforcing. This is shown in the study presented above where dermal thickness decreases (due to the emergence of my fibroblasts directly from the skin stem cells, this fibroblasts cell is the only cell that determines the elasticity cell).

B. Ophthalmologic applications

The same principle already demonstrated in the dermis happens in the eye where there are many stem cells in the lamina (the area between the cornea and sclera) on the retina and trabeculae, responsible for treating glaucoma. Due to the increased elasticity for all tissues exists also an increase in the elasticity of blood vessels and capillaries that cause losses and reduce swelling intraocular default. Thus in edema caused by the retinopathy diabetic, it is produced an increase in the tonicity of the capillaries and thus the machine according to the invention is indicated in the no proliferative diabetic retinopathy.

Clinical case 1. Patient VE, age of 59, female, was submitted on Jun. 24, 2015 at a consult and at a measurement with the Optical Coherent Tomography and it resulted that she had diabetic macular edema, FIG. 16, a).

After exposing the equipment, according to the invention for 2 hours a day for two months is observed in optical coherence tomography analyzes that swelling decreases and the retinal thickness decreases by 17% by the thickness of 422 microns to 353 microns (as in derma in the study presented in the tables above) is demonstrated by the appearance of myofibroblasts decrease the thickness of the dermis and reduce thickness). Another effect is to reduce objective the scotomas (that strain which cannot be seen in the drawings) using a device called Perimeter. These floaters are present in glaucoma and they back away to the surface, all patients demonstrated that it is a circle inside the shaded area (shaded area represents the area where there is no view across the retina represented by the great circle). See FIGS. 16, b) and c).

Clinical case 2. In patients with macular degeneration (NI) FIG. 16, and to those with Stargardt disease (CT) it is observed that the area from the retina is decreasing and it occurs an increase of the visual field objective by the perimeter.

Dry eye and dry mouth syndrome existent caused by staying on the computer or by other Sjogren syndromes are improving, that being said the patient reduces the need to use artificial tears after administration of artificial tears to 20 times a week to 5 times week. Reducing tissue elasticity in general to at the eye level determines aging the crystalline and lens feature to the presbyopia. This objectifies to the patients the delay of wearing glasses. By strengthening vascular walls it is prevented by using a specified frequency electromagnetic growth prevention of myopia, hyperopia and a delay of presbyopia.

C. Applications in Reinforcement

Clinical case 3: Male at 40 years old with mandibular dysfunction presents on the tomography computer the image of Condyle in coronal closed position that measures a thickness of the space of the articular disk of 0.7 mm. Table 13

After 200 hours of treatment to the same patient is done the same incidence and it is observed an objective thickening of the articular disc of measuring the thickness's space with the dimension of 1.5 mm. Such reinforcement cell manifests itself and symptoms of the patient are improving. Table 14.

1. An electromagnetic equipment for increasing the tissues elasticity used in the cosmetic field, contains a support (A), a coil-applicator (B) for the application of the electromagnetic field, a filter (C) for filtering the current's harmonics applied to a device (D) generating an electromagnetic field, the ensemble made up of the device (D), the filter (C) and the coil-applicator (B), being constructed to generate inside the coil-applicator (B) in an X area, an uniform electromagnetic field, with the induction between 0.7-0.8 mT, with a variation between 5-10% in the area subjected by the electromagnetic field, on a distance of 1 cm around the same point, on a larger volume in the treated areas, produced by a current of 400 mA, of a frequency between 7 and 8 Hz, preferably 7.69 Hz, with field lines perpendicular on the surface of the skin, and wherein
the coil-applicator (B) in which it applies an uniform electromagnetic field at right angles to the localized area next to their cell tissue, it is composed of two magnetic cores, one inner (5) and one outer (6) in the form of a circle and a housing (7) around each core (5, 6) being wired a small coil (9) and, respectively, a large coil (10), coupled in series with one another, so it can allow an induction of 0.6-0.8 mT in the treatment area, and a uniform magnetic field perpendicular to the treated surface, for example, the skin, the output of the large coil (10) being connected to the input of the small coil (9) and the input of the large coil (10) and the output of the small coil (9) being electrically connected to the small unit (D) electronically generating the electromagnetic field through the filter (C) so it can be filtered the harmonics current flowing in the device (D).

2. The equipment according to the claim 1, characterized in that the core (5) on which is wired the small magnetic coil (9), has a diameter of 15-33 cm, preferably 30 cm, and a thickness of 0.3-0.43 cm, preferably 0.4 cm, and the magnetic core (6) on which is wired the large magnetic coil (10), has a diameter of 16-34 cm, preferably 31.5 cm and a thickness of 0.3 0.43 cm, preferably 0.4 cm.

3. The equipment according to claim 1, characterized in that the small coil (9) is made of small enameled copper wire with a diameter of 1-1.5 mm, preferably 1.2 mm, with a number of turns between 45-50, preferably 48 turns, with a wired width of 6 cm and the large coil (10) is made of large enameled copper wire with a diameter of 1-1.5 mm, preferably 1.2 mm, the number of turns between 650-700, preferably 680 turns, with the wired width of 6 cm.

4. The equipment according to claim 1, characterized in that the assembly of the magnetic cores (5, 6), the coils (9, 10) and the filter (C) are fixed in a housing (11) made of two half-housings which consists of sticlotextolit material, which may or may not be covered by textile material for the protection of the skin.

5. The equipment according to claim 1, characterized in that the assembly of the magnetic cores (5, 6), coils (9, 10), the filter (C) and the device (D) is embedded in the housing (10).

6. The equipment according to claim 1, characterized in that the device (D) is a generator for sinusoidal current of low frequency in the range of 2 Hz-25 Hz, with the level of the output current of 400 mA, the maximum output voltage of 24V, and it is built around a microprocessor (M) which generate current signals of low frequency, which generates an electromagnetic field of intensity and uniformed induction over a distance of 1 cm around the same point, on a volume larger on areas in which would be positioned the skin, of 0.75 mT-0.8 mT, and a direct digital synthesizer (DDS) adapted to directly generate accurate sinusoidal signal with a range of 2-50 Hz, preferably 7.76 Hz, the high accuracy and stability led by the processor (M), the device D comprising a unit of processing (BP), synthesizer (DDS), a block digital amplifier (BAA) by reaction of current, an audio block amplifier (BA), a clock block (RTC) for real-time, a memory block (BM), an on/off unit (BOP) and a supply unit (ALIM).

7. The equipment according to claim 6, characterized in that the microprocessor (M) is implemented as dedicated software that has as functions:
the limitation of the usage period of the device according to the invention at the period prescribed by the medical specialist, but no more than two hours daily for a maximum of 100 hours for cosmetics to 200 hours maximum for medical applications, so it is ordered;
interruption of supply or signal generation for 12-24 hours after using for an hour or two hours daily the equipment according to the invention, depending on the desired application: cosmetic or medical;
The total interruption of the supply or signal generation after using for a maximum of 100 hours for cosmetics or up to 200 hours for medical applications
refueling the equipment or restarting the signal generation according to the invention, by a user code supplied by the manufacturer through a software implemented on the microprocessor (M).

8. Using the equipment from the claim 1 for the growth and regulation of microcirculation tissues in
nerve regeneration, for example Parkinson, dementia, depression, Alzheimer, nerve regeneration in ophthalmology
dry eye and dry mouth syndromes, adjustment of the nasal secretion
treatment for burns and wounds
treatment for hemorrhoids and varicose veins
treating genital diseases of the cervix, ovary and testicular, tissue reinforcement at the ligaments level represented by the articular disks.
regulation of the tissues microcirculation used in rhinitis, sinusitis, loss of hearing, smell, taste, sight.
